(19) United States
(12) Patent Application Publication
(10) Pub. No.: US 2024/0033529 A1
(43) Pub. Date: Feb. 1, 2024

(54) NEUROSTIMULATION SYSTEM

(71) Applicant: ADVANCED NEUROMODULATION SYSTEMS, INC., PLANO, TX (US)

(72) Inventor: Daran DeShazo, Lewisville, TX (US)

(21) Appl. No.: 18/342,412

(22) Filed: Jun. 27, 2023

Related U.S. Application Data
(60) Provisional application No. 63/369,787, filed on Jul. 29, 2022.

(51) Int. Cl. A61N 1/36; A61N 1/372

(52) U.S. Cl. CPC A61N 1/36175; A61N 1/37252

(57) ABSTRACT

Provided is an implantable pulse generator (IPG) that includes a controller, and stimulation circuitry for generating electrical pulses to stimulate neural tissue of the patient. The IPG also includes a static random-access memory (SRAM) component for storing data to control the generating of the electrical pulses, wherein the SRAM component is connected to at least the controller through a first interface bus at a first word width and is connected to at least the stimulation circuitry through a second interface bus at a second word width. The SRAM component comprises column select logic that decodes read or write (R/W) access from the controller and provides for selective connection of each column of the SRAM component to the first interface bus during the R/W access, or to the second interface bus during stimulation operations to provide stimulation control data to components of the stimulation circuitry.

### CROSS-REFERENCE TO RELATED APPLICATIONS

This application claims the benefit of U.S. Provisional Application No. 63/369,787 filed Jul. 29, 2022, titled “NEUROSTIMULATION SYSTEM”, the subject matter of which is hereby incorporated by reference in its entirety.

### BACKGROUND

Embodiments of the present disclosure generally concern neuromodulation, or neurostimulation, systems, and improvements thereof.

Implantable medical devices have improved how medical care is provided to patients with certain types of chronic illnesses and disorders. For example, implantable cardiac devices improve cardiac function in patients with heart disease thereby raising quality of life and reducing morality rates. Implantable neurostimulator can provide pain reduction for chronic pain patients and reduce motor difficulties in patients with Parkinson's disease and other movement disorders. A variety of other medical devices are proposed and are in development to treat other disorders in a wide range of patients.

Neural activity in the brain can be influenced by electrical energy that Is supplied from a stimulation pulse generator or other waveform generator, Various patient perceptions and/or neural functions can be promoted or disrupted by applying an electrical or magnetic signal to the brain. Medical researchers and clinicians have attempted to manage various neurological conditions using electrical or magnetic stimulation signals to control or affect brain functions. For example, Deep Brain Stimulation (DBS) may reduce some of the symptoms associated with Parkinson's Disease, which results in movement or muscle control problems and Is debilitating to a great number of individuals worldwide.

A stimulation system pulse generator may be provided In various configurations, such as an implanted pulse generator (IPG). A typical IPG system configuration comprises of a surgically implanted, internally-powered pulse generator and multi-electrode lead. The implanted pulse generator may commonly be encased in a hermetically sealed housing and surgically implanted, for example, in a subclavicular, upper chest, or lower back location. An electrode assembly may be implanted to deliver stimulation signals to a stimulation site. The electrode assembly is coupled to the pulse generator via biocompatible and insulated lead wires. A power source, such as a battery, is contained within the housing of the pulse generator.

Brain anatomy typically requires precise targeting of tissue for stimulation by deep brain stimulation systems. For example, deep brain stimulation for Parkinson's disease commonly targets tissue within or dose to the subthalamic nucleus (STN). The STN is a relatively small structure with diverse functions. Stimulation of undesired portions of the STN or immediately surrounding tissue can result in undesired side effects. For example, muscle contraction or muscle tightening may be caused by stimulation of neural tissue that is near the STN. Mood and behavior dysregulation and other psychiatric effects have been reported from undesired stimulation of neural tissue near the STN in Parkinson's patients.

Neuromodulation systems provide programmability of numerous parameters to optimize therapy for the condition being treated, and the individual patient. Programmable parameters include amplitude, pulse width, frequency, and electrode polarities. To represent a single stimulation pulse can take over 100 bits of data, including storage for complex waveforms of many unique pulses. Standard Static Random-Access Memories (SRAMs) can provide storage for this type of data, but has its word width set by a standard system bus (often 8, 16 or 32 bits for an embedded system) that is insufficient for other functionalities of the neuromodulation system. In addition, read and write access requires processor cycles that cost power and introduce latency. The read and write access is also constrained by limitations of direct memory access hardware, because with relation to loading stimulation data, more bits are required than can be accessed in a single word. Consequently, power is wasted in accessing memory to load stimulation data into the pulse generator. Additionally, development is made more difficult because potential race conditions must be avoided while transferring memory contents from one place to another across multiple cycles.

As a result, a need exists for a neuromodulation system that includes and improved storage for data and information.

### SUMMARY

In accordance with embodiments herein, provided is an implantable pulse generator (IPG) for providing electrical pulses to a patient for a neurostimulation therapy. The IPG includes a controller for controlling operation of the IPG, and stimulation circuitry for generating electrical pulses to stimulate neural tissue of the patient. The IPG also includes a static random-access memory (SRAM) component for storing data to control the generating of the electrical pulses, wherein the SRAM component is connected to at least the controller through a first interface bus at a first word width and is connected to at least the stimulation circuitry through a second interface bus at a second word width. The SRAM component comprises column select logic that decodes read or write (R/W) access from the controller and provides for selective connection of each column of the SRAM component to the first interface bus during the R/W access, or to the second interface bus during stimulation operations to provide stimulation control data to components of the stimulation circuitry.

Optionally, a number of columns of the SRAM component is equal to a word width of a single stimulation pulse. In one aspect, the word width of the single stimulation pulse is the first word width. In another aspect, the column select logic includes a control bit that directly connects to the first interface bus or directly connects to the second interface bus when the R/W access is decoded. In one example, the column select logic toggles between read and write access when the SRAM component is in a CPU mode. In another example, during a stimulation mode, the column select logic sets each column of the R/W access based on stimulation data requirements to allow at least one stimulation parameter and at least one diagnostic output to be within the first word width.

Optionally, the column select logic is configured to buffer data for use by the components of the stimulation circuitry. In one aspect, during the stimulation mode the SRAM component cycles through stimulation pulses by incrementing a row address so that data is available to the components of the stimulation circuitry within a single clock cycle. In another aspect, the second word length is at least 256 bits. In one example, the first interface bus is a stimulation interface bus, and the second interface bus is a central processing unit (CPU) interface bus.

In accordance with embodiments herein, provided is a computer implemented method for neurostimulation therapy. The method includes operating an implantable impulse generator (IPG) in a central processing unit (CPU) mode where a static random-access memory (SRAM) component provides read or write (R/W) access, and operating the implantable impulse generator in a stimulation mode where the SRAM component provides stimulation control data to components of stimulation circuitry during stimulation operations. The method also includes selectively connecting each column of the SRAM component to a first interface bus in the CPU mode or to a second interface bus during the stimulation mode.

Optionally, selectively connecting each column of the SRAM component includes decoding the R/W access. In one aspect, in the CPU mode, the method further comprising toggling between read and write access. In another aspect, in the stimulation mode, the method further comprising setting each column of the R/W access based on stimulation data requirements to allow at least one stimulation parameter and at least one diagnostic output to be within a first word width of the SRAM component. In one example, the method also includes buffering data for use by the components of the stimulation circuitry. In another example, in the stimulation mode, the method further comprising cycling through stimulation pulses by incrementing a row address so that data is available to the components of the stimulation circuitry within a single clock cycle.

In accordance with embodiments herein, provided is an implantable pulse generator (IPG) for providing a neurostimulation therapy. The IPG includes a controller for controlling operation of the IPG, and stimulation circuitry for generating electrical pulses to stimulate neural tissue of the patient. The IPG also includes a static random-access memory (SRAM) component for storing data to control the generating of the electrical pulses, wherein the SRAM component is connected to at least the controller through a first interface bus at a first word width and is connected to at least the stimulation circuitry through a second interface bus at a second word width. The SRAM component comprises column select logic that decodes read or write (R/W) access from the controller and provide selective connection of each column of the SRAM component to the first interface bus during the R/W access, or to the second interface bus during stimulation operations to provide stimulation control data to components of the stimulation circuitry. In addition, a word width of the single stimulation pulse is the second word width.

Optionally, the column select logic includes a control bit that directly connects to the first interface bus or directly connects to the second interface bus when the R/W access is decoded. In one aspect, the column select logic toggles between read and write access when the SRAM component is in a CPU mode. In one example, during a stimulation mode, the column select logic sets each column of the R/W access based on stimulation data requirements to allow at least one stimulation parameter and at least one diagnostic output to be within the first word width.

### BRIEF DESCRIPTION OF THE DRAWINGS

FIG. 1 depicts a neurostimulation system that is adapted according to an example embodiment and is shown as a high-level functional block diagram.

FIG. 2 depicts a controller for a neurostimulation system and is shown as a high-level functional block diagram.

FIG. 3 depicts a schematic diagram of a static random-access memory according to an example embodiment.

FIG. 4 depicts a flow chart illustrating a method operating a neurostimulation system.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

The term “static random-access memory component” or “SRAM component” as used herein shall refer to a memory, storage device, etc. that stores data, information, etc. until power is lost to the SRAM component. In one example, the SRAM component stores the data, information, etc. in bits that are arranged in columns and rows. In another example, the SRAM component includes latch circuitry to store the bits.

The term “word width” when used herein refers to the number of digits, or bits in a line of code. Example word widths can include 16 bits, 32 bits, 64 bits, 128 bits, 256 bits, etc.

The term “read or write”, “read/write”, or “R/W” when used herein refers to a device, component, media, or the like that can have data read from the device, component, media, or the like, and data written to the device, component, media, or the like. In particular, the device, component, media, or the like includes properties to allow both reading and writing functionality.

Provided is a neurostimulation system that includes SRAM architecture that allows the read and write path to be configured to communicate with a central processing unit (CPU) interface bus of one word width and a stimulation interface bus of another word width. A SRAM component includes an array of six transistor bit cells with a single word width that can access an entire row of memory. In addition, the total number of columns is set to equal the width of the word width required to represent a single stimulation pulse. In one example the stimulation pulse width can be 256 bits. The column select logic can then have a control bit that allows each column to connect directly to the stimulation pulse wide (e.g. 256-bit) bus for stimulation control. In addition, the column can be decoded to connect to a 16 or 32-bit wide bus for an alternative purpose, such as processor read/write access. In particular, the columns are toggled between read and write access when the memory is in CPU mode, and when stimulation is running, columns are set to be in a read or write (R/W) state based on the stimulation data needs allowing stimulation parameters and diagnostic outputs to be contained in the same word. Data that is being read out of the memory is buffered by the column select logic and can be used directly by the stimulation circuits like register outputs. Cycling through stimulation pulses in a program thus consists of incrementing the row address, and data is available to stimulation circuits within a single clock cycle. In one example, the neurostimulation system can even be running on a low-frequency stimulation, or on a real time clock.

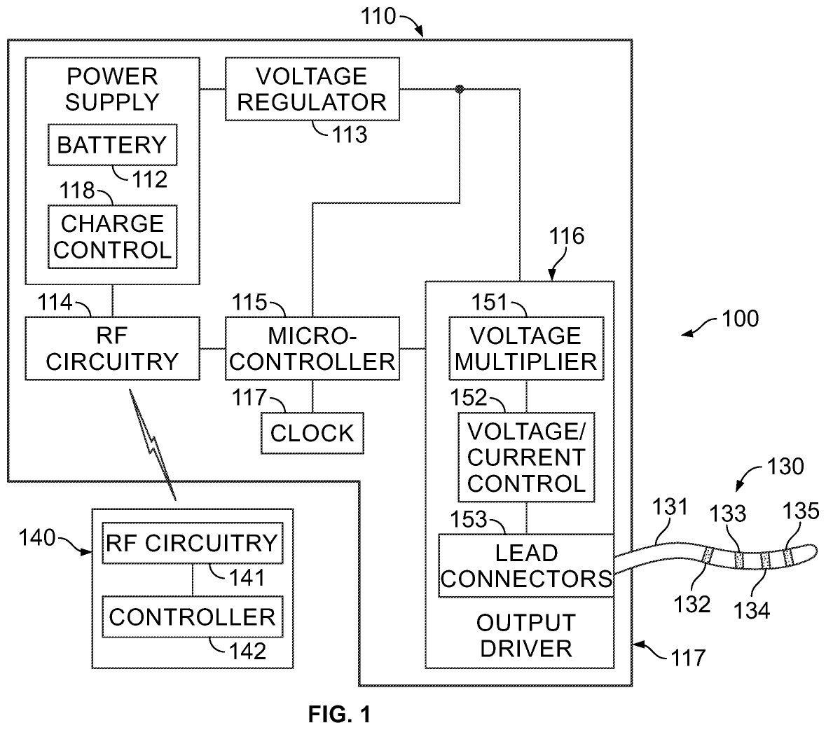

FIG. 1 depicts a neurostimulation system 100 that is adapted according to an example embodiment and is shown as a high-level functional block diagram. Neurostimulation systems are devices that generate electrical pulses and deliver the pulses to neural tissue of a patient to treat a variety of disorders. As noted above, a neurostimulation system 100 may be used to provide DBS therapy for patients with movement disorders. Neurostimulation system 100 may also provide Spinal Cord Stimulation (SCS) in which electrical pulses are delivered to neural tissue of the spinal cord for the purpose of chronic pain control. While a precise understanding of the interaction between the applied electrical energy and the neural tissue is not fully appreciated, it is known that application of an electrical field to spinal neural tissue can effectively inhibit certain types of pain transmitted from regions of the body associated with the stimulated neural tissue to the brain.

Neurostimulation systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. For SCS therapy, the distal end of a respective stimulation lead is implanted within the epidural space to deliver the electrical pulses to the appropriate nerve tissue within the spinal cord. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

The neurostimulation system 100 of the illustrated embodiment includes a generator portion, shown as implantable pulse generator (IPG) 110, for providing a stimulation or energy source, a stimulation portion, shown as lead 130, for application of the stimulus pulse(s), and an optional external controller, shown as programmer/controller 140, to program and/or control IPG 110 via a wireless communications link. IPG 110 may be implanted within a living body (not shown) for providing electrical stimulation from IPG 110 to a selected area of the body, such as a region of the brain or spinal cord, via lead 130. In some embodiments, IPG 110 provides electrical stimulation under control of external programmer/controller 140. It should be appreciated that, although lead 130 is illustrated to provide a stimulation portion of stimulation system 100 and is configured to provide stimulation remotely with respect to the generator portion 110 of stimulation system 100, a lead 130 as described herein is intended to encompass a variety of stimulation portion configurations. Furthermore, a lead configuration may include more (e.g., 8, 16, 32, etc.) or fewer (e.g., 1, 2, etc.) electrodes than those represented in the illustrations.

The IPG 110 of the illustrated embodiment includes power supply 111, voltage regulator 113, RF circuitry 114, microcontroller (or microprocessor) 115, stimulation circuitry 116, and clock 117, as are described in further detail below. A power supply 111 provides a source of power, such as from battery 112, to other components of the IPG 110, as may be regulated by a voltage regulator 113. The battery 112 may comprise a non-rechargeable (e.g., single use) battery, a rechargeable battery, a capacitor, and/or like power sources, Charge control 118 provides management for battery 112 and power supply 111 in some embodiments. In some embodiments, the entire IPG 110 device may need to be accessed by a surgical procedure to replace battery 112. In other embodiments, when battery 112 is depleted, it may be recharged after being implanted, for example, inductive coupling and external charging circuits. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

RF circuitry 114 provides data communication between microcontroller 115 and controller 142 in external programmer/controller 140, via RF circuitry 141. It should be appreciated that RF circuitry 114 and/or 141 may be a receiver, a transmitter, and/or transceiver depending upon the communication links desired using far-field and/or near field communication communications. The communication links may be established using suitable communication methods such as inductive wireless communication, low energy BLUETOOTH® communication, and medical band wireless communication as examples. An example of BLUETOOTH@ communication between an implantable medical device and a programmer device is found, for example, in U.S. Pat. No. 9,894,691, entitled SYSTEMS AND METHODS FOR ESTABLISHING A COMMUNICATION LINK BETWEEN AN IMPLANTABLE MEDICAL DEVICE AND AN EXTERNAL INSTRUMENT, the disclosure of which is incorporated herein by reference.

Microcontroller 115 provides control with respect to the operation of IPG 110, such as in accordance with a program provided thereto by external programmer/controller 140. Software code is typically stored in memory (not shown) of IPG 110 for execution by the microcontroller 115 to control the various components of the device. The software code stored in memory of IPG 110 may support operations of embodiments disclosed herein.

Stimulation circuitry 116 generates and delivers pulses to selected ones of electrodes 132-135 on lead body 131 under control of microcontroller 115. The stimulation circuitry 116 includes numerous components 151, 152, 153, or the like for performing the operations of the IPG 110. For example, voltage multiplier 151 and voltage/current control 152 may be controlled to deliver a constant current pulse of a desired magnitude using selected ones of electrodes 132-135. Clock 117 preferably provides system timing information, such as may be used by microcontroller 115 in controlling system operation, as may be used by voltage multiplier 151 in generating a desired voltage, etcetera.

Lead 130 of the illustrated embodiment includes lead body 131, preferably incorporating a plurality of internal conductors coupled to lead connectors (not shown) to interface with lead connectors 153 of IPG 110. Lead 130 further includes electrodes 132-135, which are preferably coupled to the internal conductors 153. The internal conductors provide electrical connection from individual lead connectors to each of a corresponding one of electrodes 132-235. In the exemplary embodiment the lead 130 is generally configured to transmit one or more electrical signals from IPG 110 for application at, or proximate to, a spinal nerve or peripheral nerve, brain matter, muscle, or other tissue via electrodes 132-135. IPG 110 is capable of controlling the electrical signals by varying signal parameters, such as pulse amplitude, pulse width, pulse frequency, burst frequency, and/or the like in order to deliver a desired therapy or otherwise provide operation as described herein.

Although the embodiment illustrated in FIG. 1 includes four electrodes, it should be appreciated that any number of electrodes, and corresponding conductors, may be utilized according to some embodiments. Moreover, various types, configurations, and shapes of electrodes (and lead connectors) may be used according to some embodiments. An optional lumen (not shown) may extend through the lead 130, such as for use in delivery of chemicals or drugs or to accept a stylet during placement of the lead within the body. Additionally, or alternatively, the lead 130 (stimulation portion) and IPG 110 (generator portion) of stimulation system 100 may comprise a unitary construction, such as that of a microstimulator configuration.

In an embodiment, a programmable neurostimulation system 100 supplies suitable therapy pulses to a patient by enabling a pattern of electrical pulses to be varied across the electrodes 132-135 of a lead or leads 130. Such systems enable electrodes of a connected stimulation lead 130 to be set as an anode(+), as a cathode(−), or to a high-impedance state (OFF). As is well known, negatively charged ions flow away from a cathode toward an anode. Consequently, a range of very simple to very complex electrical fields can be created by defining different electrodes 132-135 in various combinations of (+), (−), and OFF. Of course, in any instance, a functional combination must include at least one anode and at least one cathode. In an embodiment, the case or “can” of the neurostimulation system 100 or IPG 110 may function as an anode. When determining the appropriate electrode configurations, the selection of electrodes 132-135 to function as anodes can often facilitate isolation of the applied electrical field to desired fibers and other neural structures. Specifically, the selection of an electrode 132-135 to function as an anode at a position adjacent to another electrode functioning as a cathode causes the resulting electron/ion flow to be limited to tissues immediately surrounding the two electrodes. By alternating through the possible anode/cathode combinations, it is possible to gain greater resolution in the stimulation of desired tissue or neural structures.

As mentioned above, programmer/controller 140 provides data communication with IPG 110, such as to provide control (e.g., adjust stimulation settings), provide programming (e.g., alter the electrodes to which stimulation pulses are delivered), etc. Accordingly, programmer/controller 140 of the illustrated embodiment includes RF circuitry 141 for establishing a wireless link with IPG 110, and controller 142 to provide control with respect to IPG 110. Programmer/controller 140 may receive data from IPG 110 that can be displayed to medical personnel or a clinician on a screen (not shown) on programmer/controller 140. Additionally, or alternatively, programmer/controller 140 may provide power to IPG 110, such as via RF transmission by RF circuitry 141. Optionally, however, a separate power controller may be provided for charging the power source 111 within IPG 110.

Additional detail with respect to pulse generation systems and the delivery of stimulation pulses may be found in U.S. Pat. No. 6,609,031, entitled “MULTIPROGRAMMABLE TISSUE STIMULATOR AND METHOD,” the disclosure of which is hereby incorporated herein by reference. Similarly, additional detail with respect to pulse generation systems and the delivery of stimulation pulses may be found in U.S. Pat. No. 7,937,158, entitled “MULTIPROGRAMMABLE TRIAL STIMULATOR,” the disclosure of which is hereby incorporated herein by reference. Also, additional detail with respect to pulse generation system and delivery of stimulation pulses may be found in U.S. Pat. Pub. No. 2021/0402192, entitled “IMPLANTABLE PULSE GENERATOR FOR PROVIDING A NEUROSTIMULATION THERAPY USING COMPLEX IMPEDANCE MEASUREMENTS AND METHODS OF OPERATION,” the disclosure of which is hereby incorporated herein by reference.

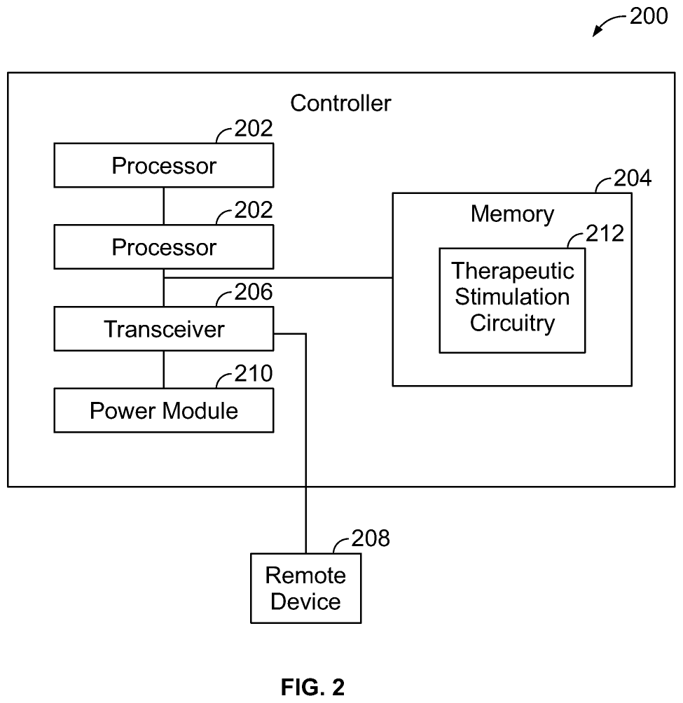

FIG. 2 illustrates a schematic diagram of example controller 200 of an IPG. In one example, the controller 200 of FIG. 2 is the controller 142 of FIG. 1. The controller 200 can include one or more processors 202, a memory 204, a transceiver 206 for communicating with remote devices 208, a power module 210, and stimulation circuitry 212. The memory 204 in one example is a static random-access memory (SRAM) component that retains data and information until the controller is powered off. In another example, the stimulation circuitry 212 includes components, including components for implanting the stimulation.

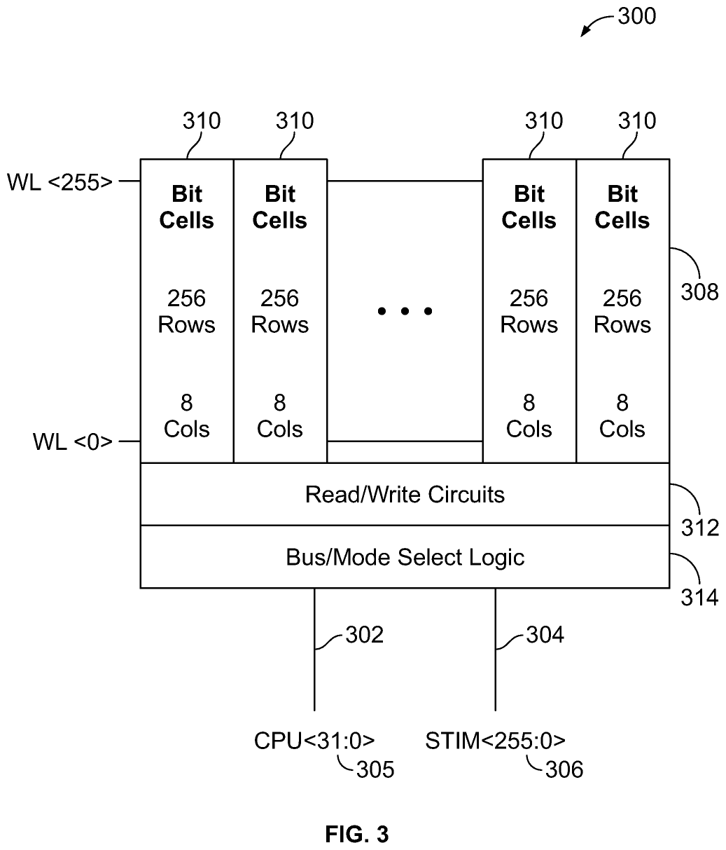

FIG. 3 illustrates one example of a SRAM component 300 that can be the memory 204 of FIG. 2. In the example embodiment of FIG. 3 the SRAM component 300 is a 256×256 bit memory (e.g. 256 big columns and 256 bit rows). The SRAM component 300 architecture allows the RAN path to be configured to a first interface bus 302 at a first word width 305. In one example, the first interface bus 302 bus is a central processing unit (CPU) interface bus (of the one or more processors 202). In the example embodiment of FIG. 3, the CPU interface bus is shown having a first word width of <31:0>, that indicates a 32 bit width.

In addition, the SRAM component 300 includes a second interface bus 304 at a second word width 306. In one example, the second interface bus 304 is a stimulation interface bus (of the stimulation circuitry 212). In the example embodiment of FIG. 3, the second word width of <255:0> is provided, indicating a 256 bit word width. To this end, in this example embodiment, the first word width 305 (e.g. 32 bits) is different than the second word width 306 (e.g. 256 bits).

The SRAM component architecture provides column select logic 314 for selective connection of each column of the SRAM component to the first interface bus or to the second interface bus. In particular, the column select logic 314 can decode R/W access from the controller and provide selective connection of each column of the SRAM component to the first interface bus during the R/W access, or to the second interface bus during stimulation operations to provide stimulation control data to components of the stimulation circuitry. In one example, a control bit of the column select logic 314 provides the selective connection. In one example, the selective connection can directly connect each column of the SRAM component to the first interface bus during R/W access, or directly connect each column of the SRAM component to the second interface bus during stimulation operations to provide stimulation control data to components of the stimulation circuitry. In this manner, when the SRAM component is operating a first mode, or a CPU mode, the SRAM component is connected to the first interface, and in a second mode, or stimulation mode, the SRAM component is connected to the second interface. In addition, while in the CPU mode (e.g. when R/W access is provided), the column select logic 314 can toggle between read and write access.

In the example of FIG. 3, the SRAM component 300 includes 256 rows 308, along with 32 columns 310 that are each 8 bits wide to provide the 256×256 bit configuration. To this end, the SRAM component includes an array of six-transistor bit cells (not illustrated), and a first word width (e.g. 32) that accesses an entire row 308 of memory 300. In addition, the total number of bits in a column 310 (e.g. 256) is set to equal the second word width 306, that in one example is the word width required to represent a single stimulation pulse when the SRAM component 300 is operating in a stimulation mode. In one example the width of the second word width 306 for the single stimulation pulse is 256 bits. Alternatively, the word width 306 could be 64 bits, 128 bits, 324 bits, 512 bits, or the like.

The SRAM component 300 also includes R/W circuits 312 that include column select logic 314 with a control bit that allows each column 310 to connect directly using the second word width 306 of the second interface bus 304 (e.g. 256 bit width of a stimulation interface bus), or the second word width 306 can be decoded to provide the first word width 305 to connect to the first interface bus 302 (e.g. the CPU interface bus having a first word width of only 16 bits, 32 bits, 64 bits etc.) that has a smaller word width than the second interface bus 304. The connection to the first interface bus 302 in the embodiment where the first interface bus 302 is a CPU interface bus allows for efficient read and/or write functionality. When coupled to a CPU interface bus, or within a CPU, the columns can be toggled as needed between read and write access.

In one example, when the SRAM component 300 is operating in a stimulation mode, the columns 310 can be set to either R/W based on stimulation data need. As a result, the stimulation parameter and diagnostic outputs can be contained within the second word width 306. In particular, during the stimulation mode, the column select logic 314 can set each column of the R/W access based on stimulation data requirements to allow at least one stimulation parameter and at least one diagnostic output to be within the first word width. In addition, data that is being read out of the memory can be buffered by the column select logic and can be used directly by the stimulation circuitry (e.g. 212) to function as a register output. Cycling through the stimulation pulses in a program thus consists of incrementing the row address and data that is available to the stimulation circuitry (e.g. 212) within a single clock cycle. In one example, the stimulation pulse provides low-frequency stimulation, or a real time clock can be provided.

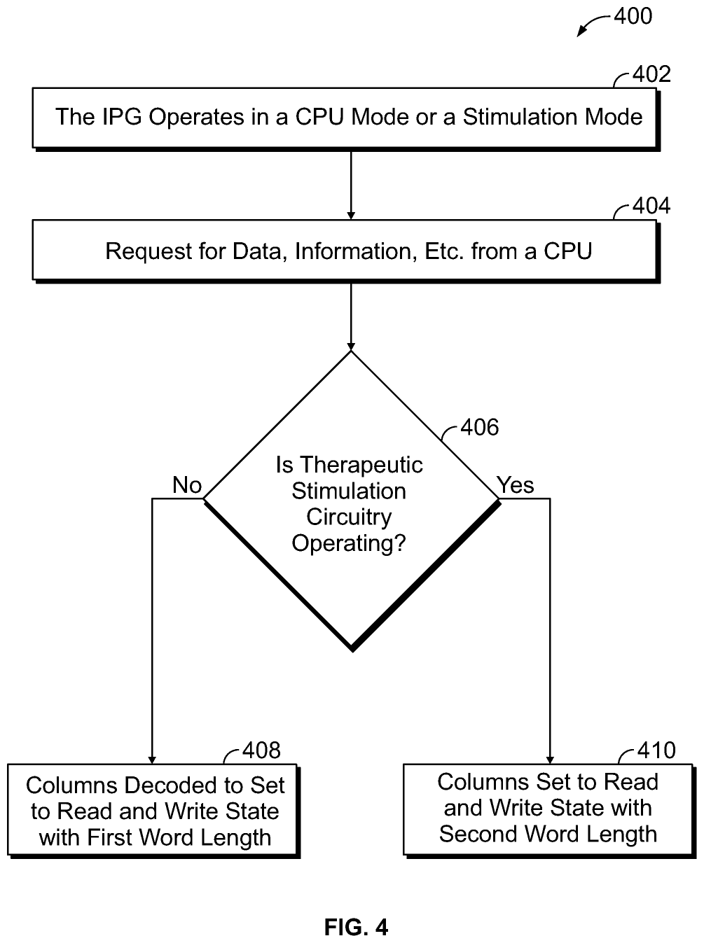

FIG. 4 depicts a block flow diagram of a method 400 for operating an IPG. In one example, the IPG of FIG. 1 with the controller of FIG. 2, and SRAM component of FIG. 3 are utilized to implement and/or perform the method 400. By performing the method 400, power cost is improved, and latency reduced over IPGs that do not have the SRAM component as provided herein.

At 402, the IPG operates in a CPU mode or a stimulation mode. In the CPU mode, stimulation is not occurring, and column select logic decodes the columns to provide a first word width. The first word width is selected to be a word width that matches a word width of a first interface bus, or CPU interface bus. In one example, the first word width is 32 bits. In the stimulation mode a SRAM component includes a column that is a second word width that interfaces with a second interface bus that can be a stimulation interface bus of stimulation circuitry. In an example, the second word width can be 256 bits. In this manner, the column is provided to interface with the stimulation circuitry in order to provide the width needed for stimulation.

At 404, a request is made for data, information, etc. from a CPU. In response to the request, at 406, a determination is made whether the stimulation circuitry is operating. If at 406, the stimulation circuitry is not operating, then at 408, column select logic includes a control bit that decodes the columns to provide a first word length that matches a word length of a first interface bus (e.g. a CPU interface bus) to provide R/W access for the processor. In example embodiments, the first word length can be 16 bits, 32 bits, etc. By decoding the columns to match the first interface bus (e.g. CPU interface bus), latency is reduced when R/W functionality is desired.

If at 406, the stimulation circuitry is operating, then at 410 the columns are set in a R/W state based on the stimulation data needs. This allows stimulation parameters and diagnostic outputs to be contained within the same row that uses the second word width. In this manner, during a stimulation mode, column select logic can set each column of the R/W access based on stimulation data requirements to allow at least one stimulation parameter and at least one diagnostic output to be within the first word width. In an example embodiment, the second word length can be 128 bits, 256 bits, etc. In particular, the first word length is less than the second word length.

Thus provided is a SRAM component optimized for storage of stimulation parameters and diagnostic outputs. The word width in the SRAM component can be set independently for CPU access or stimulation access based on the operating state of the neuromodulation system. Consequently, digital data is available 100% of the time during the operation of the neuromodulation system, including when the neuromodulation system is in a stimulation mode. The RAN direction is set per column of an SRAM component during the stimulation mode so that diagnostic data is contained in the same row as input parameters for the stimulation. To this end, multiple unique pulses are provided in a compact SRAM component structure, while a CPU can access write stimulation memory and read stimulation memory. In particular, the stimulation mode can operate from the SRAM component without waking up or utilizing the CPU, or using a fast clock. As a result power is saved. In addition, stimulation input and output data can all be accessed at one time, eliminating race condition, and simplifying development. Therefore, problems of the Background are overcome.

One or more of the operations described above in connection with the methods may be performed using one or more processors. The different devices in the systems described herein may represent one or more processors, and two or more of these devices may include at least one of the same processors. In one embodiment, the operations described herein may represent actions performed when one or more processors (e.g., of the devices described herein) execute program instructions stored in memory (for example, software stored on a tangible and non-transitory computer readable storage medium, such as a computer hard drive, ROM, RAM, or the like).

The processor(s) may execute a set of instructions that are elements may also store data or other information as desired or needed. The storage element may be in the form of an information source or a physical memory element within the controllers and the controller device. The set of instructions may include various commands that instruct the controllers and the controller device to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

The controller may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. When processor-based, the controller executes program instructions stored in memory to perform the corresponding operations. Additionally, or alternatively, the controllers and the controller device may represent circuits that may be implemented as hardware. The above examples are exemplary only and are thus not intended to limit in any way the definition and/or meaning of the term “controller”.

### Closing Statements

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method, or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices, and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An implantable pulse generator (IPG) for providing electrical pulses to a patient for a neurostimulation therapy, comprising:
a controller for controlling operation of the IPG;
stimulation circuitry for generating electrical pulses to stimulate neural tissue of the patient; and
a static random-access memory (SRAM) component for storing data to control the generating of the electrical pulses, wherein the SRAM component is connected to at least the controller through a first interface bus at a first word width and is connected to at least the stimulation circuitry through a second interface bus at a second word width;
wherein the SRAM component comprises column select logic that decodes read or write (R/W) access from the controller and provides for selective connection of each column of the SRAM component to the first interface bus during the R/W access, or to the second interface bus during stimulation operations to provide stimulation control data to components of the stimulation circuitry.

2. The IPG of claim 1, wherein a number of columns of the SRAM component is equal to a word width of a single stimulation pulse.

3. The IPG of claim 2, wherein the word width of the single stimulation pulse is the first word width.

4. The IPG of claim 1, wherein the column select logic includes a control bit that directly connects to the first interface bus or directly connects to the second interface bus when the R/W access is decoded.

5. The IPG of claim 1 wherein the column select logic toggles between read and write access when the SRAM component is in a CPU mode.

6. The IPG of claim 1, wherein during a stimulation mode, the column select logic sets each column of the R/W access based on stimulation data requirements to allow at least one stimulation parameter and at least one diagnostic output to be within the first word width.

7. The IPG of claim 1, wherein the column select logic is configured to buffer data for use by the components of the stimulation circuitry.

8. The IPG of claim 1, wherein during the stimulation mode the SRAM component cycles through stimulation pulses by incrementing a row address so that data is available to the components of the stimulation circuitry within a single clock cycle.

9. The IPG of claim 1, wherein the second word length is at least 256 bits.

10. The IPG of claim 1, wherein the first interface bus is a stimulation interface bus, and the second interface bus is a central processing unit (CPU) interface bus.

11. A computer implemented method for neurostimulation therapy comprising:
operating an implantable impulse generator (IPG) in a central processing unit (CPU) mode where a static random-access memory (SRAM) component provides read or write (R/W) access;
operating the implantable impulse generator in a stimulation mode where the SRAM component provides stimulation control data to components of stimulation circuitry during stimulation operations; and
selectively connecting each column of the SRAM component to a first interface bus in the CPU mode or to a second interface bus during the stimulation mode.

12. The method of claim 11, wherein selectively connecting each column of the SRAM component includes decoding the R/W access.

13. The method of claim 11, wherein in the CPU mode, the method further comprising toggling between read and write access.

14. The method claim 11, wherein in the stimulation mode, the method further comprising setting each column of the R/W access based on stimulation data requirements to allow at least one stimulation parameter and at least one diagnostic output to be within a first word width of the SRAM component.

15. The method of claim 11, further comprising buffering data for use by the components of the stimulation circuitry

16. The method of claim 11, wherein in the stimulation mode, the method further comprising cycling through stimulation pulses by incrementing a row address so that data is available to the components of the stimulation circuitry within a single clock cycle.

17. An implantable pulse generator (IPG) for providing a neurostimulation therapy, comprising:
a controller for controlling operation of the IPG;
stimulation circuitry for generating electrical pulses to stimulate neural tissue of the patient; and
a static random-access memory (SRAM) component for storing data to control the generating of the electrical pulses, wherein the SRAM component is connected to at least the controller through a first interface bus at a first word width and is connected to at least the stimulation circuitry through a second interface bus at a second word width;
wherein the SRAM component comprises column select logic that decodes read or write (R/W) access from the controller and provide selective connection of each column of the SRAM component to the first interface bus during the R/W access, or to the second interface bus during stimulation operations to provide stimulation control data to components of the stimulation circuitry;
wherein a word width of the single stimulation pulse is the second word width.

18. The IPG of claim 17, wherein the column select logic includes a control bit that directly connects to the first interface bus or directly connects to the second interface bus when the R/W access is decoded.

19. The IPG of claim 17 wherein the column select logic toggles between read and write access when the SRAM component is in a CPU mode.

20. The IPG of claim 17, wherein during a stimulation mode, the column select logic sets each column of the R/W access based on stimulation data requirements to allow at least one stimulation parameter and at least one diagnostic output to be within the first word width.
